SEQUENCE LISTING (20 sequences)

SEQ ID NO: 1 - DNA, 441 bp, Homo sapiens
  CDS (1)..(441): Hu-Leptin
gtgcccatccaaaaagtccaagatgacaccaaaaccctcatcaagacaattgtcaccaggatcaatgacatttcacacacgcagtcagtctcctccaaacagaaagtcaccggtttggacttcattcctgggctccaccccatcctgaccttatccaagatggaccagacactggcagtctaccaacagatcctcaccagtatgccttccagaaacgtgatccaaatatccaacgacctggagaacctccgggatcttcttcacgtgctggccttctctaagagctgccacttgccctgggccagtggcctggagaccttggacagcctggggggtgtcctggaagcttcaggctactccacagaggtggtggccctgagcaggctgcaggggtctctgcaggacatgctgtggcagctggacctcagccctgggtgctga

SEQ ID NO: 2 - protein, 146 aa, Homo sapiens
VPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 3 - DNA, 441 bp, Mus musculus
  CDS (1)..(441): Mu-Leptin
gtgcctatccagaaagtccaggatgacaccaaaaccctcatcaagaccattgtcaccaggatcaatgacatttcacacacgcagtcggtatccgccaagcagagggtcactggcttggacttcattcctgggcttcaccccattctgagtttgtccaagatggaccagactctggcagtctatcaacaggtcctcaccagcctgccttcccaaaatgtgctgcagatagccaatgacctggagaatctccgagacctcctccatctgctggccttctccaagagctgctccctgcctcagaccagtggcctgcagaagccagagagcctggatggcgtcctggaagcctcactctactccacagaggtggtggctttgagcaggctgcagggctctctgcaggacattcttcaacagttggatgttagccctgaatgctga

SEQ ID NO: 4 - protein, 146 aa, Mus musculus
VPIQKVQDDTKTLIKTIVTRINDISHTQSVSAKQRVTGLDFIPGLHPILSLSKMDQTLAVYQQVLTSLPSQNVLQIANDLENLRDLLHLLAFSKSCSLPQTSGLQKPESLDGVLEASLYSTEVVALSRLQGSLQDILQQLDVSPEC

SEQ ID NO: 5 - DNA, 696 bp, Homo sapiens
  CDS (1)..(696): HuFc
gagcccaaatcttctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcacgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 6 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - DNA, 699 bp, Mus musculus
  CDS (1)..(699): MuFc
gagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggaccccgggtaaa

SEQ ID NO: 8 - protein, 233 aa, Mus musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - DNA, 29 bp, Artificial Sequence
  Description of Artificial Sequenceforward primer to clone and adapt the murine leptin cDNA
cccgggtaaagtgcctatccagaaagtcc

SEQ ID NO: 10 - DNA, 27 bp, Artificial Sequence
  Description of Artificial Sequencereverse primer to clone and adapt the murine leptin cDNA
ctcgagtcagcattcagggctaacatc

SEQ ID NO: 11 - DNA, 30 bp, Artificial Sequence
  Description of Artificial Sequenceforward primer to clone and adapt human leptin cDNA
cccgggtaaagtgcccatccaaaaagtcca

SEQ ID NO: 12 - DNA, 10 bp, Artificial Sequence
  Description of Artificial Sequencecarboxy terminus of the immunoglobulin heavy chain
cccgggtaaa

SEQ ID NO: 13 - DNA, 27 bp, Artificial Sequence
  Description of Artificial Sequencereverse primer to clone and adapt human leptin cDNA
ctcgagtcagcacccagggctgaggtc

SEQ ID NO: 14 - DNA, 27 bp, Artificial Sequence
  Description of Artificial Sequenceforward primer to adapt murine leptin cDNA
cttaagcgtgcctatccagaaagtcca

SEQ ID NO: 15 - DNA, 24 bp, Artificial Sequence
  Description of Artificial Sequencereverse primer to adapt murine leptin cDNA
gatatcgcattcagggctaacatc

SEQ ID NO: 16 - DNA, 11 bp, Artificial Sequence
  Description of Artificial SequenceEcoRI/AflII linker-adaptor
aattcgatatc

SEQ ID NO: 17 - DNA, 11 bp, Artificial Sequence
  Description of Artificial SequenceEcoRI/AflII linker-adaptor
ttaagatatcg

SEQ ID NO: 18 - DNA, 24 bp, Artificial Sequence
  Description of Artificial Sequencelinker
ggcgcaggaggttctggcggatcc

SEQ ID NO: 19 - DNA, 24 bp, Artificial Sequence
  Description of Artificial Sequencelinker
ggatccgccagaacctcctgcgcc

SEQ ID NO: 20 - protein, 146 aa, Artificial Sequence
  Description of Artificial Sequenceconsensus leptin sequence
VPXXXXQDDTKTLIKTIVXRINDISHTXSVSXXQXVXGLDFIPGLXPXLXLSXMDQTLAXYQQXLXXXXSXNXXQIXXDLENLRDLLHXLAXSKSCXLPXXXXLXXXXSLXXVLEASXYSTEVVALSRLQXXLQDXLXXLDXSPXC

(19) United States
(12) Patent Application Publication
(10) Pub. No.: US 2004/0053366 A1
(43) Pub. Date: Mar. 18, 2004

(54) Expression and export of anti-obesity proteins as Fc fusion proteins

(75) Inventors: Kin-Ming Lo, Lexington, MA (US); Jinyang Zhang, Arlington, MA (US); Stephen D. Gillies, Carlisle, MA (US)

Correspondence Address: TESTA, HURWITZ & THIBEAULT, LLP, HIGH STREET TOWER, 125 HIGH STREET, BOSTON, MA, 02110, US

(73) Assignee: Lexigen Pharmaceuticals Corp., Lexington, MA (US)

(21) Appl. No.: 10/419,058

(22) Filed: Apr. 18, 2003

Related U.S. Application Data
(63) Continuation of application No. 09/479,508, filed on Jan. 7, 2000, now abandoned.
(60) Provisional application No. 60/115,079, filed on Jan. 7, 1999.

(51) Int. Cl. C12P 21/02; C12P 21/06; C07H 21/04; C12N 5/06; C07K 16/18

(52) U.S. Cl. 435/69.1; 435/320.1; 435/326; 530/387.1; 536/23.53

(57) ABSTRACT

Disclosed are nucleotide sequences, for example, DNA or RNA sequences, which encode an immunoglobulin Fc-Leptin fusion protein. The nucleotide sequences can be inserted into a suitable expression vector and expressed in mammalian cells. Also disclosed is a family of immunoglobulin Fc-Leptin fusion proteins that can be produced by expression of such nucleotide sequences. Also disclosed are methods using such nucleotide sequences and fusion proteins for treating conditions which are alleviated by the administration of leptin.

### RELATED APPLICATIONS

This application claims priority to U.S. Provisional Application Serial No. 60/115,079, filed Jan. 7, 1999, the disclosure of which is incorporated herein by reference.

### FIELD OF THE INVENTION

The present invention relates generally to methods and compositions for making and using fusion proteins containing an anti-obesity protein. More particularly, the invention relates to methods and compositions for making and using fusion proteins which contain an immunoglobulin Fc region and a leptin anti-obesity protein.

### BACKGROUND OF THE INVENTION

Obesity is a major physiological disorder associated with a number of maladies such as diabetes, hypertension, heart disease and certain types of cancers. In the United States, it is estimated that more than 30% of the adult population is obese, i.e., at least 20% over ideal body weight. There are also increasing indications that obesity is fast becoming a serious health problem worldwide. It is recognized that in many cases diet and exercise alone are insufficient to achieve a reduction in body weight, especially in people who inherit genetic traits that predispose them to becoming obese. There is, therefore, a need for a drug that can help people lose weight and lower the risks of obesity-related disorders. More specifically, there is a need for an anti-obesity drug with enough potency to cause substantial weight loss at feasible dose levels. Because obesity is defined as being 20% over ideal weight, a weight loss of at least 20% is desirable. In more severe cases, a weight loss of 30-60% can be necessary to bring a person's weight down into a healthy range.

Obesity is a multifactorial phenotype, which may result from a combination of physiological, psychological, genetic and environmental factors. One factor associated with obesity is the obese (ob) gene which has now been cloned (Zhang et al. (1994) NATURE 372:425). In normal mice, the ob gene encodes a hormone called leptin (Friedman et al. (1998) NATURE 395:763). In a satiated state, excess energy is converted and stored as triglycerides in adipocytes, which in turn secrete leptin into the blood stream. Leptin functions as a messenger by binding to its receptor, a long form of which has a cytoplasmic domain capable of signal transduction and is found predominantly in the hypothalamus. It is contemplated that hormone-receptor binding is a signaling mechanism through which the adipose tissue can inform the brain about the status of energy stores. It is contemplated that leptin crosses the blood-brain barrier to gain access to leptin receptors located in the hypothalamus (Spiegelman et al. (1996) CELL 87:377). When the brain receives a message that the energy stores are plentiful, it tells the body to adjust accordingly, by reducing food intake and/or increasing energy expenditure.

A strain of morbidly obese mice referred to as ob/ob mice are homozygotes having two mutant ob alleles. The mutant alleles produce truncated leptin, which is non-functional and probably degrades rapidly in vivo. Consequences of leptin deficiency in ob/ob mice include lethargy, hypothermia, hyperglycemia, hyperinsulinemia, and infertility. In humans, there is also evidence associating weight gain and obesity to leptin deficiency (Montague et al. (1997) NATURE 387:903; Ravussin et al. (1997) NATURE MEDICINE 3:238), although it has been reported that the majority of obese people have high levels of circulating leptin (Considine et al. (1995) N. ENGL. J. MED. 334:292).

Symptoms associated with leptin deficiency in ob/ob mice can be ameliorated by the administration of recombinant leptin. Daily intraperitoneal injections of leptin can reduce food intake, body weight, percent body fat, and serum concentrations of glucose and insulin. This was accompanied by increases in metabolic rate, body temperature and locomotor activities, all of which require energy expenditure (Pelleymounter et al. (1995) SCIENCE 269:540; Halaas et al. (1995) SCIENCE 269:543). In the same studies, normal mice also benefited from leptin treatment, although the reductions in body weight, food intake and body fat were significantly smaller. Recombinant leptin also has been used to correct infertility in both female and male ob/ob mice (Chebab et al. (1996) NATURE GENETICS 12:318; Mounzib et al. (1997) ENDOCRINOLOGY 138:1190). Furthermore, recent experiments using transgenic mice suggested that about 5 to 10% of obese humans having relatively normal or low leptin levels may be responsive to leptin treatment (Ioffe et al. (1998) PROC. NATL. ACAD. SCI. USA 95:11852).

The use of leptin in its present forms requires high doses of the protein to be injected multiple times daily for months to achieve the desired clinical outcome. For example, in a recent clinical trial, some volunteers on the high dose range required leptin to be injected three times daily for six months (WALL STREET JOURNAL, Jun. 15, 1998). Presumably, frequent, high doses are needed due to a combination of low potency and short serum half-life of leptin. This observation also is consistent with observations in ob/ob mouse models in which an intraperitoneal injection of 5 to 20 mg/kg/day of leptin was needed to demonstrate a significant reduction in body weight (Pelleymounter et al. (1995) SCIENCE 269:540; Hallas et al. (1995) SCIENCE 269:543; Chebab et al. (1996) NATURE GENETICS 12:318; Mounzih et al. (1997) ENDOCRINOLOGY 138:1190). To overcome the “suboptimal pharmacokinetics” of leptin, a chronic infulsion of leptin at 400 ng/hr subcutaneously was needed to achieve a physiologic plasma level of leptin in mice (Halaas et al. (1997) PROC. NATL. ACAD. SCI. USA 94:8878).

Major reasons for the frequent, high doses appear to be due to one or more intrinsic properties, for example, size, of leptin and the method by which the pharmacological agent was prepared. Leptin has a molecular weight of about 16 kD (Halaas et al. (1 995) SCIENCE 269:543) and thus is small enough to be cleared by renal filtration. Hence a high dose may be necessary to compensate for the short serum half life in vivo.

Moreover, smaller proteins such as leptin can be produced in bacteria, for example, E. coli. Under certain circumstances, the recombinant leptin is produced as insoluble inclusion bodies in E. coli. Prior to use, the inclusion bodies must be solubilized with a denaturing agent, for example, guanidine hydrochloride, purified under denaturing conditions, and folded under appropriate conditions to produce functional protein. In addition, leptin contains two cysteine residues which participate in an intramolecular disulfide bond. Thus, to maximize the recovery of a soluble, biologically active molecule, the folding process needs to be controlled carefully to minimize the formation of insoluble protein aggregates and intermolecular disulfide bonds.

As a result of such a complicated production process, i.e., leptin purified from inclusion bodies made in prokaryotes, it may not be possible to provide a well-defined homogeneous protein sample with full biological activity. Attempts to improve the solubility of leptin have included mutating certain amino acid residues to aspartates or glutamates thereby lowering the isoelectric point (pI) of leptin from 5.84 to below 5.5 (U.S. Pat. No. 5,719,266). Although such manipulation results in a product that can be more readily formulated and stored, the product also is a mutant protein which could be immunogenic in the intended recipient.

Given the high dosage, low efficacy, short serum half-life, and very complex processes involved in the production and purification of leptin, there is a need in the art for methods of enhancing the production and improving the pharmacological properties of this anti-obesity agent.

### SUMMARY OF THE INVENTION

The present Invention features methods and compositions useful for making and using fusion proteins containing an anti-obesity protein, for example, leptin. The fusion proteins can facilitate high level expression of biologically active anti-obesity proteins. The fusion protein can be combined with a pharmaceutically acceptable carrier prior to administration to a mammal, for example, a human. Under certain circumstances, the anti-obesity protein can be cleaved from the fusion protein prior to formulation and/or administration. Alternatively, nucleic acid sequences encoding the anti-obesity protein containing fusion protein can be combined with a pharmaceutically acceptable carrier and administered to the mammal.

It is an object of the invention to provide novel nucleic acid sequences, for example, DNAs and RNAs, which facilitate the production and secretion of leptin. In particular, objects of the invention are (i) to provide novel nucleic acid sequences which facilitate efficient production and secretion of leptin; (ii) to provide nucleic acid constructs for the rapid and efficient production and secretion of leptin in a variety of mammalian host cells; and (iii) to provide methods for the production, secretion and collection of recombinant leptin or genetically engineered variants thereof, including non-native, biosynthetic, or otherwise artificial leptin proteins such as proteins which have been created by rational design.

Other objects of the invention are to provide polynucleotide sequences which, when fused to a polynucleotide encoding leptin, encode a leptin containing fusion polypeptide which can be purified using common reagents and techniques. Yet another object is to interpose a proteolytic cleavage site between a secretion cassette and the encoded leptin protein such that the secretion cassette can be cleaved from the leptin domain so leptin may be purified independently.

Another object of the invention is to provide fusion proteins containing leptin. The fusion proteins of the present invention demonstrate improved biological properties over native leptin such as increased solubility, prolonged serum half-life and increased binding to its receptor. These properties may improve significantly the clinical efficacy of leptin. In a preferred embodiment, the fusion protein comprises, in an N- to C-terminal direction, an immunoglobulin Fc region and leptin, with other moieties, for example, a proteolytic cleavage site, optionally interposed between the immunoglobulin Fc region and the leptin. The resulting fusion protein preferably is synthesized in a cell that glycosylates the Fc region at normal glycosylation sites, i.e., which usually exist in template antibodies. Glycosylation contributes, at least in part, to the enhanced circulatory half-life of the fusion protein.

Other objects of the invention are to provide multivalent and multimeric forms of leptin fusion proteins, and combinations thereof.

Another object of the invention is to provide methods of treatment using the fusion proteins, or cleaved leptin. An overall object of the invention is to provide processes which are both efficient and inexpensive as well as yield biologically active anti-obesity proteins.

Accordingly, in one aspect, the present invention provides nucleic acid molecules, for example, DNA or RNA molecules, which encode an immunoglobulin Fc region-leptin fusion protein. The nucleic acid molecule encodes a signal sequence, an immunoglobulin Fc region, and at least one target protein, also referred to herein as the anti-obesity protein, for example, leptin. In a preferred embodiment, the nucleic acid molecule encodes, serially in a 5′ to 3′ direction, the signal sequence, the immunoglobulin Fc region and the target protein sequence. In another embodiment, the nucleic acid molecule encodes, serially in a 5′ to 3′ direction, the signal sequence, the target sequence, and the immunoglobulin Fc region. The nucleic acid may encode an X-Fc or Fc-X structure where X is a target protein such as leptin. The preferred embodiments are the Fc-X structures because of their superior level of expression.

In a preferred embodiment, the immunoglobulin Fc region comprises an immunoglobulin hinge region and preferably comprises at least one immunoglobulin constant heavy region domain, for example, an immunoglobulin constant heavy 2 (CH2) domain, an immunoglobulin constant heavy 3 (CH3) domain, and depending upon the type of immunoglobulin used to generate the Fc region, optionally an immunoglobulin constant heavy chain 4 (CH4) domain. In a more preferred embodiment, the immunoglobulin Fc region lacks at least an immunoglobulin constant heavy 1 (CH1) domain. Although the immunoglobulin Fc regions may be based on any immunoglobulin class, for example, IgA, IgD, IgE, IgG, and IgM, immunoglobulin Fc regions based on IgG are preferred.

The nucleic acid of the invention can be incorporated in operative association into a replicable expression vector which can then be introduced into a mammalian host cell competent to produce the leptin-based fusion protein. The resultant leptin-based fusion protein is produced efficiently and secreted from the mammalian host cell. The secreted leptin-based fusion protein may be collected from the culture media without lysing the mammalian host cell. The protein product can be assayed for activity and/or purified using common reagents as desired, and/or cleaved from the fusion partner, all using conventional techniques.

In another aspect, the invention provides a fusion protein comprising an immunoglobulin Fc region linked, either directly through a polypeptide bond or indirectly via a polypeptide linker, to the target protein. The target protein may be fused via its C-terminal end to an N-terminal end of the immunoglobulin Fc region. However, in a more preferred embodiment the target protein is fused via its N-terminal end to a C-terminal end of the immunoglobulin Fc region.

In one embodiment, the fusion proteins of the invention when administered at a dose of about 0.25 mg/kg/day for 5 days to an ob/ob mouse having an initial body weight of at least about 50 grams, induce about a 10% (about 5 gram), more preferably about a 12% (about 6 gram) or more preferably about a 15% (about 7.5 gram) loss of the initial body weight. In a more preferred embodiment, the fusion proteins of the invention, when administered at a dose of about 0.1 mg/kg/day for 5 days to an ob/ob mouse having an initial body weight of at least about 50 grams, induce about a 10% (about 5 gram), more preferably about a 12% (about 6 gram), or more preferably about a 15% (about 7.5 gram) loss of the initial body weight.

In another embodiment, the fusion protein may comprise a second target protein, for example, mature, full length leptin or a bioactive fragment thereof. In this type of construct the first and second target proteins can be the same or different proteins. The first and second target proteins may be linked together, either directly or by means of a polypeptide linker. Alternatively, both target proteins may be linked either directly or via a polypeptide linker, to the immunoglobulin Fc region. In the latter case, the first target protein can be connected to an N-terminal end of the immunoglobulin Fc region and the second target protein can be connected to a C-terminal end of the immunoglobulin Fc region.

In another embodiment, two fusion proteins may associate, either covalently, for example, by a disulfide or polypeptide bond, or non-covalently, to produce a dimeric protein. In a preferred embodiment, the two fusion proteins are associated covalently by means of at least one and more preferably two interchain disulfide bonds via cysteine residues, preferably located within immunoglobulin hinge regions disposed within the immunoglobulin Fc regions of each chain.

In another aspect, the invention provides methods of producing a fusion protein comprising an immunoglobulin Fc region and the target protein. The method comprises the steps of (a) providing a mammalian cell containing a DNA molecule encoding such a fusion protein, either with or without a signal sequence, and (b) culturing the mammalian cell to produce the fusion protein. The resulting fusion protein can then be harvested, refolded, if necessary, and purified using conventional purification techniques well known and used in the art. Assuming that the fusion protein comprises a proteolytic cleavage site disposed between the immunoglobulin Fc region and the target protein, the target can be cleaved from the fusion protein using conventional proteolytic enzymes and if necessary, purified prior to use.

In yet another aspect, the invention provides methods for treating conditions lo alleviated by leptin or active variants thereof by administering to a mammal an effective amount of leptin produced by a method of the invention and/or a fusion construct of the invention. The invention also provides a method for treating conditions alleviated by leptin or active variants thereof by administering a DNA or RNA of the invention, for example, a “naked DNA,” or a vector containing a DNA or RNA of the invention, to a mammal having the condition.

The foregoing and other objects, features and advantages of the present invention will be made more apparent from the detailed description, drawings, and claims that follow.

### BRIEF DESCRIPTION OF THE DRAWINGS

FIGS. 1A-1E are schematic illustrations of exemplary anti-obesity fusion proteins constructed in accordance with the invention. The Figures depict, respectively, FIG. 1A, dimeric Fc-leptin; FIG. 1B, dimeric Fc-leptin-GlySer linker leptin fragment; FIG. 1C, dimeric Fc-leptin-GlySer linker-leptin; FIG. 1D, dimeric leptin-Fc; and FIG. 1E, dimeric leptin-GlySer linker-Fc. The vertical lines represent optional disulfide bonds connecting cysteine residues (C) disposed within a hinge region of each immunoglobulin region.

FIG. 2 is a graph showing the body weight of ob/ob mice in grams treated with IP injections of 0.25 mg/kg of muLeptin-linker-muFc (diamonds), 0.25 mg/kg muLeptin-muFc (squares), 0.25 mg/kg muFc-MuLeptin (triangles), or phosphate buffered saline (PBS) (crosses).

FIG. 3 is a graph showing the body weight of ob/ob mice treated with daily (daily for the first 12 days, and thereafter only Monday through Friday) intraperitoneal (IP) injections of either 0.25 mg/kg of muFc-muLeptin (diamonds) or phosphate-buffered saline (PBS) (squares).

FIG. 4 is a graph showing the body weight of ob/ob mice in grams treated with daily intravenous (IV) injections of 0.25 mg/kg of muFc-muLeptin (triangles), 1.0 mg/kg muFc-muLeptin (circles), or PBS (squares) for five days, followed by no treatment.

FIG. 5 is a graph showing the effect of different dosing schedules on the body weight of ob/ob mice treated with subcutaneous (SC) injections of muFc-muLeptin (0.25 mg/kg (diamonds); and 0.1 mg/kg followed by 1.0 mg/kg (squares)) or PBS (triangles).

FIG. 6 is a graph showing the body weight of ob/ob mice in grams treated with intraperitoneal (IP) injections of 0.1 mg/kg of huFc-huLeptin (diamonds), 0.5 mg/kg huFc-huLeptin (squares), or PBS (triangles).

FIG. 7 is a graph showing the circulating levels in serum of glycosylated huFc-huLeptin (diamonds) and unglycosylated huFc (N→Q mutation)-huLeptin (squares) as a function of time (hours) post administration. The circulating levels are expressed as a percentage of the initial dose.

### DETAILED DESCRIPTION OF THE INVENTION

The invention provides fusion proteins which are useful in the production of anti-obesity proteins. The fusion proteins of the invention and/or nucleic acids encoding such fusion proteins may be administered directly to mammals in need of treatment with an anti-obesity protein. It is contemplated, however, that the anti-obesity proteins may be cleaved from the fusion proteins prior to use.

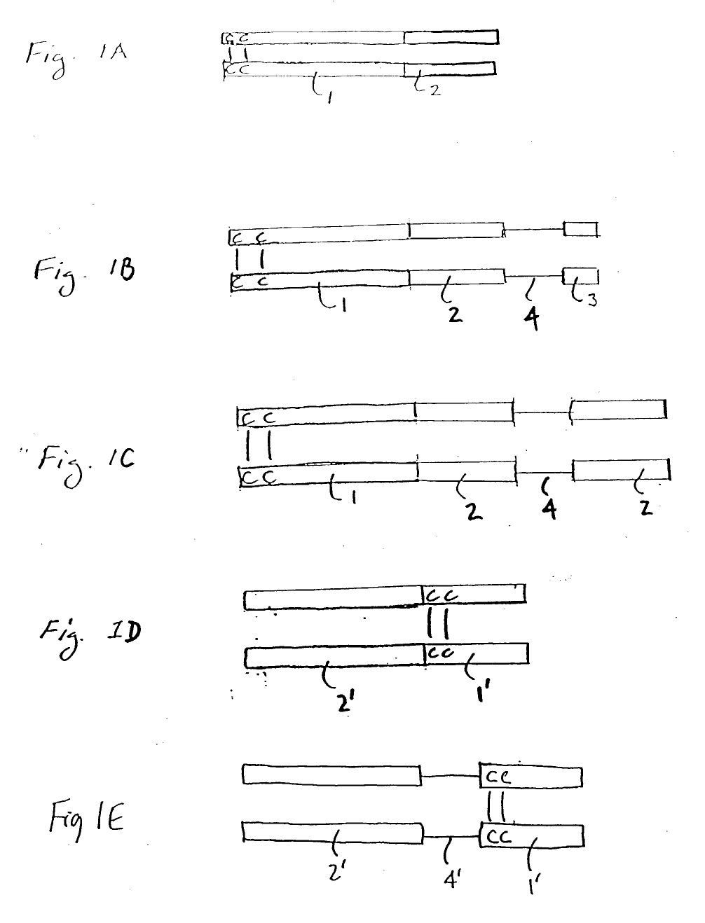

The invention thus provides fusion proteins comprising an immunoglobulin Fc region and at least one target protein, referred to herein as leptin. Five exemplary embodiments of protein constructs embodying the invention are illustrated in the drawing as FIGS. 1A-1E. Because dimeric constructs are preferred, all are illustrated as dimers cross-linked by a pair of disulfide bonds between cysteines in adjacent subunits. In the drawings, the disulfide bonds are depicted as linking together the two immunoglobulin heavy chain Fc regions via an immunoglobulin hinge region within each heavy chain, and thus are characteristic of native forms of these molecules. While constructs including the hinge region of Fc are preferred and have been shown promise as therapeutic agents, the invention contemplates that the crosslinking at other positions may be chosen as desired. Furthermore, under some circumstances, dimers or multimers useful in the practice of the invention may be produced by non-covalent association, for example, by hydrophobic interaction.

Because homodimeric constructs are important embodiments of the invention, the drawings illustrate such constructs. It should be appreciated that heterodimeric structures also are useful in the practice of the invention. However, viable constructs useful to inhibit obesity in various mammalian species including humans can be constructed, e.g., one chain of a dimeric Fc fusion protein comprising a full length leptin and the other chain of the dimeric Fc fusion protein comprising a leptin variant.

FIG. 1A illustrates a dimeric construct produced in accordance with the principles set forth herein (see, for example, Examples 1 and 4). Example 1 expresses the murine construct and Example 4 expresses the human construct. Each monomer of the homodimer comprises an immunoglobulin Fc region I including a hinge region, a CH2 domain and a CH3 domain. Attached directly, i.e., via a polypeptide bond, to the C terminus of the Fc region is leptin 2. It should be understood that the Fc region may be attached to a target protein via a polypeptide linker (not shown).

FIGS. 1B and 1C depict protein constructs of the invention which include as a target protein plural anti-obesity proteins arranged in tandem and connected by a linker. In FIG. 1B, the target protein comprises full length leptin 2, a polypeptide linker made of glycine and serine residues 4, and an active variant of leptin 3. FIG. 1C differs from the construct of FIG. 1B in that the most C-terminal protein domain comprises a second full length copy of leptin 2.

Although FIGS. 1A-1C represent Fc-X constructs, where X is the target protein, it is contemplated that X-Fc type constructs may also be useful in the practice of the invention. Accordingly, FIGS. 1D and 1E depict X-Fc-type constructs made in accordance with the principles set forth herein (see, for example, Examples 5 and 6). The X-Fc-type construct depicted in FIG. 1D comprises, at its N-terminus, a full length leptin 2′. Connected directly to the leptin's C-terminus is an Fc region 1′ including a hinge region. In FIG. 1E, the illustrated construct has at its N-terminus a full length leptin 2′. In contrast to the construct of FIG. 1D, however, the leptin 2′ depicted in FIG. 1E is connected by a polypeptide linker 4′ to an Fc region 1′. Furthermore, it is contemplated that useful proteins of the invention may also be depicted by the formula X-Fc-X, wherein the X's may represent the same or different target proteins.

As used herein, the term “polypeptide linker” is understood to mean a peptide sequence that can link together two proteins that in nature are not naturally linked together. The polypeptide linker preferably comprises a plurality of amino acids such as alanine, glycine and serine or combinations of such amino acids. Preferably, the polypeptide linker comprises a series of glycine and serine peptides about 10-15 residues in length. See, for example, U.S. Pat. No. 5,258,698, the disclosure of which is incorporated herein by reference. It is contemplated, however, that the optimal linker length and amino acid composition may be determined by routine experimentation.

As used herein, the term “multivalent” refers to a recombinant molecule that incorporates two or more biologically active segments. The protein fragments forming the multivalent molecule may be linked through a polypeptide linker which attaches the constituent parts and permits each to function independently.

As used herein, the term “bivalent” refers to a multivalent recombinant molecule having the configuration Fc-X or X-Fc, where X is a target molecule. The immunoglobulin Fc regions can associate, for example, via interchain disulfide bonds, to produce the type of constructs shown in FIGS. 1A and 1D. If the fusion construct of the invention has the configuration Fc-X—X, the resulting Fc dimer molecule is shown in FIG. 1C. The two target proteins may be linked through a peptide linker. Constructs of the type shown in FIG. 1A can increase the apparent binding affinity between the target molecule and its receptor. For instance, if one leptin moiety of an Fc-Leptin fusion protein can bind to a receptor on a cell with a certain affinity, the second leptin moiety of the same Fc-Leptin fission protein may bind to a second receptor on the same cell with a much higher avidity (apparent affinity). This may occur because of the physical proximity of the second leptin moiety to the receptor after the first leptin moiety already is bound. In the case of an antibody binding to an antigen, the apparent affinity may be increased by at least ten thousand-fold, i.e., 10⁴. Each protein subunit, i.e., “X,” has its own independent function so that in a multivalent molecule, the functions of the protein subunits may be additive or synergistic.

As used herein, the term “multimeric” refers to the stable association of two or more polypeptide chains either covalently, for example, by means of a covalent interaction, for example, a disulfide bond, or non-covalently, for example, by hydrophobic interaction. The term multimer is intended to encompass both homomultimers, wherein the subunits are the same, as well as, heteromultimers, wherein the subunits are different.

As used herein, the term “dimeric” refers to a specific multimeric molecule where two polypeptide chains are stably associated through covalent or non-covalent interactions. It should be understood that the immunoglobulin Fc region including at least a portion of the hinge region, a CH2 domain and a CH3 domain, typically forms a dimer. Many protein ligands are known to bind to their receptors as a dimer. If a protein ligand X dimerizes naturally, the X moiety in an Fc-X molecule will dimerize to a much greater extent, since the dimerization process is concentration dependent. The physical proximity of the two X moieties connected by Fc would make the dimerization an intramolecular process, greatly shifting the equilibrium in favor of the dimer and enhancing its binding to the receptor.

As used herein, the term “leptin” is understood to mean not only full length mature leptin protein (see, for example, SEQ ID NO: 2 and SEQ ID NO: 4 which represent mature human leptin and murine leptin, respectively), but also variants and bioactive fragments thereof. The term bioactive fragment refers to any leptin protein fragment that has at least 30%, more preferably at least 70%, and most preferably at least 90% of the biological activity of the mature, template leptin protein, as determined using the ob/ob mouse model. The term variants includes species and allelic variants, as well as other naturally occurring or non-naturally occurring variants, for example, generated by genetic engineering protocols, that are at least 70% similar or 60% identical, more preferably at least 75% similar or 65% identical, and most preferably at least 80% similar or 70% identical to either the naturally-occurring sequences of leptin disclosed herein.

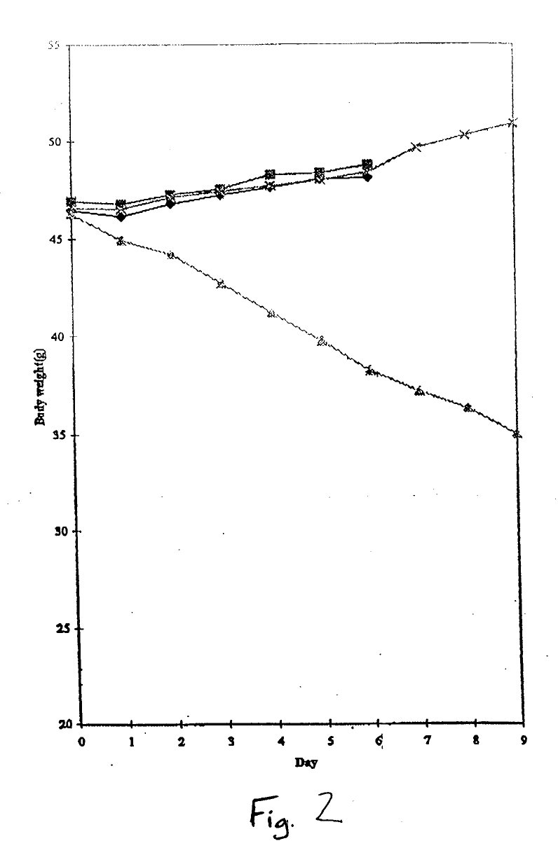

To determine whether a candidate polypeptide has the requisite percentage similarity or identity to a reference polypeptide, the candidate amino acid sequence and the reference amino acid sequence are first aligned using the dynamic programming algorithm described in Smith and Waterman (1981) J. MOL. BIOL. 147:195-197, in combination with the BLOSUM62 substitution matrix described in FIG. 2 of Henikoff and Henikoff (1992), “Amino acid substitution matrices from protein blocks”, PROC. NATL. ACAD. SCI. USA 89:10915-10919. For the present invention, an appropriate value for the gap insertion penalty is −12, and an appropriate value for the gap extension penalty is −4. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and reference sequence is made, a percent similarity score may be calculated. The individual amino acids of each sequence are compared sequentially according to their similarity to each other. If the value in the BLOSUM62 matrix corresponding to the two aligned amino acids is zero or a negative number, the pair-wise similarity score is zero; otherwise the pair-wise similarity score is 1.0. The raw similarity score is the sum of the pair-wise similarity scores of the aligned amino acids. The raw score then is normalized by dividing it by the number of amino acids in the smaller of the candidate or reference sequences. The normalized raw score is the percent similarity. Alternatively, to calculate a percent identity, the aligned amino acids of each sequence again are compared sequentially. If the amino acids are non-identical, the pair-wise identity score is zero; otherwise the pair-wise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity. Accordingly, gap penalties are not used in this calculation, although they are used in the initial alignment.

Variants may also include other leptin muteins having leptin-like activity. See, for example, U.S. Pat. No. 5,719,266, the disclosure of which is incorporated by reference herein. Species variants, include, but are not limited to human and mouse leptin sequences (see, for example, SEQ ID NOS 2 and 4, respectively) and the species variants encoded by nucleotide sequences disclosed in the Genbank and/or EMBL databases, for example, under accession numbers U72873 (Pongo pygmaeus), U96450 (Pan froglogytes), U66254 (Sus scrota), U50365 (Bos taurus), D49653 (Rattus norvegicus), U58492 (Macaca mulatta), U72872 (Gorilla gorilla), U62123 (Ovis aries), AF082500 (Gallus gallus), AF082501 (Meleagris gallopavo), ABO20986 (Canis familiaris), AF097582 (Equus caballus), and AF 159713 (Sminihopsis crassicaudata), the disclosures of which are incorporated herein by reference.

Furthermore, the leptin sequence may comprise a portion or all of the consensus sequence set forth in SEQ ID NO: 20, wherein the leptin has at least 30%, more preferably at least 70%, and most preferably at least 90% of the biological activity of mature, full length human leptin, as determined using the ob/ob mouse model. The consensus sequence of SEQ ID NO: 20, was generated from leptin sequences derived from mouse, rat, chicken, human, chimpanzee, cow, sheep, lowland gorilla, rhesus monkey, pig, orangutang and dog. For example, the leptin may comprise a portion or all of the consensus sequence:

```
Val Pro Xaa Xaa Xaa Xaa Gln Asp Asp Thr Lys Thr Leu Ile Lys Thr
    1 5 10 15
Ile Val Xaa Arg Ile Asn Asp Ile Ser His Thr Xaa Ser Val Ser Xaa
                          20 25 30
Xaa Gln Xaa Val Xaa Gly Leu Asp Phe Ile Pro Gly Leu Xaa Pro Xaa
                  35 40 45
Leu Xaa Leu Ser Xaa Met Asp Gln Thr Leu Ala Xaa Tyr Gln Gln Xaa
          50 55 60
Leu Xaa Xaa Xaa Xaa Ser Xaa Asn Xaa Xaa Gln Ile Xaa Xaa Asp Leu
  65 70 75 80
Gln Asn Leu Arg Asp Leu Leu His Xaa Leu Ala Xaa Ser Lys Ser Cys
                                  85 90 95
Xaa Leu Pro Xaa Xaa Xaa Xaa Leu Xaa Xaa Xaa Xaa Ser Leu Xaa Xaa
                        100 105 110
Val Leu Glu Ala Ser Xaa Tyr Ser Thr Glu Val Val Ala Leu Ser Arg
                115 120 125
Leu Gln Xaa Xaa Leu Gln Asp Xaa Leu Xaa Xaa Leu Asp Xaa Ser Pro
        130 135 140
Xaa Cys
145
```

(SEQ ID NO: 20), wherein optionally Xaa3 can be Ile or Cys, Xaa4 can be Arg, Trp, Gln or His, Xaa5 can be Lys, Arg, or Ile, Xaa6 can be Val or Phe, Xaa19 can be Ala or Thr, Xaa28 can be Gln or a peptide bond, Xaa32 can be Ser or Ala, Xaa33 can be Lys or Arg, Xaa35 can be Arg or Lys, Xaa37 can be Ala or Thr, Xaa46 can be Gin or His, Xaa48 can be Val, Ile or Lys, Xaa50 can be Ser or Thr, Xaa53 can be Arg, Lys or Gin, Xaa60 can be Ile or Val, Xaa64 can be Ile or Val, Xaa66 can be Ans, Thr, Ile, or Ala, Xaa67 xan be Leu or Met, Xaa68 can be Leu or Met, Xaa69 can be His or Pro, Xaa7l can be Arg or Gln, Xaa73 can be Val or Met, Xaa74 can be Val, Ile or Leu, Xaa77 can be Ser or Ala, Xaa78 can be Asn or His, Xaa89 can be Leu or Val, Xaa92 can be Ser, Phe or Ala, Xaa97 can be Pro, His or Ser, Xaa100 can be Arg, Qln, Trp or Leu, Xaa101 can be Ala, Val or Thr, Xaa102 can be Arg or Ser, Xaa103 can be Gly or Ala, Xaa105 can be Glu or Gln, Xaa106 can be Thr, Ser or Lys, Xaa107 can be Phe, Leu or Pro, Xaa108 can be Glu or Asp, Xaa111 can be Gly or Asp, Xaa112 can be Gly, Asp or Val, Xaa118 can be Leu or Gly, Xa131 can be Ala, Gly or Arg, Xaa132 can be Ala or Ser, Xaa136 can be Met or Ile, Xaa 138 can be Arg, Trp or Qln, Xaa139 can be Arg or Gln, Xaa142 can be Leu or Val, or Xaa145 can be Gly or Glu.

In preferred embodiments, the target protein includes the full length, mature sequence of leptin. The nucleotide sequences encoding and the amino acid sequences defining human and murine leptin proteins are set forth in SEQ ID NOS: 1-4.

The target proteins disclosed herein are expressed as fusion proteins with an Fc region of an immunoglobulin. As is known, each immunoglobulin heavy chain constant region comprises four or five domains. The domains are named sequentially as follows: CH1-hinge-CH2-CH3(-CH4). The DNA sequences of the heavy chain domains have cross-homology among the immunoglobulin classes, e.g., the CH2 domain of IgG is homologous to the CH2 domain of IgA and IgD, and to the CH3 domain of IgM and IgE.

As used herein, the term, “immunoglobulin Fc region” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In a preferred embodiment the immunoglobulin Fc region comprises at least an immunoglobulin hinge region a CH2 domain and a CH3 domain, and preferably lacks the CH1 domain.

The currently preferred class of immunoglobulin from which the heavy chain constant region is derived is IgG (Igγ) (γ subclasses 1, 2, 3, or 4). The nucleotide and amino acid sequences of human Fc γ-1 are set forth in SEQ ID NOS: 5 and 6. The nucleotide and amino acid sequences of murine Fc γ-2a are set forth in SEQ ID NOS: 7 and 8. Other classes of immunoglobulin, IgA (Igα), IgD (Igδ), IgE (Igε) and IgM (Igμ), may be used. The choice of appropriate immunoglobulin heavy chain constant regions is discussed in detail in U.S. Pat. Nos. 5,541,087, and 5,726,044. The choice of particular immunoglobulin heavy chain constant region sequences from certain immunoglobulin classes and subclasses to achieve a particular result is considered to be within the level of skill in the art. The portion of the DNA construct encoding the immunoglobulin Fc region preferably comprises at least a portion of a hinge domain, and preferably at least a portion of a CH₃ domain of Fcγ or the homologous domains in any of IgA, IgD, IgE, or IgM.

Depending on the application, constant region genes from species other than human, for example, mouse or rat may be used. The immunoglobulin Fc region used as a fusion partner in the DNA construct generally may be from any mammalian species. Where it is undesirable to elicit an immune response in the host cell or animal against the Fc region, the Fc region may be derived from the same species as the host cell or animal. For example, a human immunoglobulin Fc region can be used when the host animal or cell is human; likewise, a murine immunoglobulin Fc region can be used where the host animal or cell will be a mouse.

Nucleic acid sequences encoding, and amino acid sequences defming human and murine immunoglobulin Fc regions useful in the practice of the invention are set forth in SEQ ID NOS: 5-8. However, it is contemplated that other immunoglobulin Fc region sequences useful in the practice of the invention may be found, for example, by those encoded by nucleotide sequences disclosed in the Genbank and/or EMBL databases, for example, AF045536.1 (Macaca fuscicularis), AF045537.1 (Macaca mulatta), AD016710 (Felix cat), K00752 (Oryctolagus cuniculus), U03780 (Sus scrofa), Z48947 (Camelus dromedarius), X62916 (Bos taurus), L07789 (Mustela vison), X69797 (Ovis aries), U17166 (Cricetulus migratorius), X07189 (Ratius rattus), AF57619.1 (Trichosurus vulpecuila), or AF035195 (Monodelphis dioestica), the disclosures of which are incorporated by reference herein.

Furthermore, it is contemplated that substitution or deletion of amino acids within the immunoglobulin heavy chain constant regions may be useful in the practice of the invention. One example would be to introduce amino acid substitutions in the upper CH2 region to create a Fc variant with reduced affinity for Fc receptors (Cole et al. (1997) J. IMMUNOL. 159:3613). One of ordinary skill in the art can prepare such constructs using well known molecular biology techniques.

The use of human Fcγ1 as the Fc region sequence has several advantages. For example, if the Fc fusion protein is to be used as a biopharmaceutical, the Fcγ1 domain may confer effector function activities to the fusion protein. The effector function activities include the biological activities such as placental transfer and increased serum half-life. The immunoglobulin Fc region also provides for detection by anti-Fc ELISA and purification through binding to Staphylococcus aureus protein A (“Protein A”). In certain applications, however, it may be desirable to delete specific effector functions from the immunoglobulin Fc region, such as Fc receptor binding and/or complement fixation.

In the fusion proteins of the invention, the immunoglobulin Fc regions facilitate proper folding of the leptin protein to yield active leptin proteins and also impart solubility to the active moieties, at least in the extracellular medium. Since the immunoglobulin Fc region is hydrophilic, the leptin containing fusion protein is soluble unlike the leptin counterparts expressed in a bacterial host. DiMarchi et al. (U.S. Pat. No. 5,719,266) improved the solubility of leptin by mutating certain amino acid residues to aspartates or glutamates, thereby lowering the isoelectric point (pI) of leptin from 5.84 to below 5.5. The use of the immunoglobulin Fc region as a fusion partner reduces the need for creation of leptin muteins with a lower pI, because Fc is glycosylated and highly charged at physiological pI, and hence acts as a carrier to solubilize leptin As a result, leptin containing fusion protein is completely soluble in aqueous solutions, for example, pharmaceutically acceptable carriers.

It is understood that the present invention exploits conventional recombinant DNA methodologies for generating the Fc fusion proteins useful in the practice of the invention. The Fc fusion constructs preferably are generated at the DNA level, and the resulting DNAs integrated into expression vectors, and expressed to produce the fusion proteins of the invention. As used herein, the term “vector” is understood to mean any nucleic acid comprising a nucleotide sequence competent to be incorporated into a host cell and to be recombined with and integrated into the host cell genome, or to replicate autonomously as an episome. Such vectors include linear nucleic acids, plasmids, phagemids, cosmids, RNA vectors, viral vectors and the like. Non-limiting examples of a viral vector include a retrovirus, an adenovirus and an adeno-associated virus. As used herein, the term “gene expression” or “expression” of a target protein, is understood to mean the transcription of a DNA sequence, translation of the mRNA transcript, and secretion of an Fc fusion protein product.

A useful expression vector is pdCs (Lo et al. (1988) PROTEIN ENGINEERING 11:495, the disclosure of which is incorporated herein by reference) in which the transcription of the Fc-X gene utilizes the enhancer/promoter of the human cytomegalovirus and the SV40 polyadenylation signal. The enhancer and promoter sequence of the human cytomegalovirus used was derived from nucleotides −601 to +7 of the sequence provided in Boshart et al. (1985) CELL 41:521, the disclosure of which is incorporated herein by reference. The vector also contains the mutant dihydrofolate reductase gene as a selection marker (Simonsen and Levinson (1983) PROC. NAT. ACAD. SCI. USA 80:2495, the disclosure of which is incorporated herein by reference).

An appropriate host cell can be transformed or transfected with the DNA sequence of the invention, and utilized for the expression and/or secretion of the target protein. Currently preferred host cells for use in the invention include immortal hybridoma cells, NS/O myeloma cells, 293 cells, Chinese hamster ovary cells, HELA cells, and COS cells.

One expression system that has been used to produce high level expression of fusion proteins in mammalian cells is a DNA construct encoding, in the 5′ to 3′ direction, a secretion cassette, including a signal sequence and an immunoglobulin Fc region, and a target protein. Several target proteins have been expressed successfully in such a system and include, for example, IL2, CD26, Tat, Rev, OSF-2, βIG-H3, IgE Receptor, PSMA, and gp120. These expression constructs are disclosed in U.S. Pat. Nos. 5,541,087 and 5,726,044 to Lo et al., the disclosures of which are incorporated by reference herein.

As used herein, the term “signal sequence” is understood to mean a segment which directs the secretion of the leptin fusion protein and thereafter is cleaved following translation in the host cell. The signal sequence of the invention is a polynucleotide which encodes an amino acid sequence which initiates transport of a protein across the membrane of the endoplasmic reticulum. Signal sequences which are useful in the invention include antibody light chain signal sequences, e.g., antibody 14.18 (Gillies et. al. (1989) J. IMMUNOL. METH. 125:191), antibody heavy chain signal sequences, e.g., the MOPC141 antibody heavy chain signal sequence (Sakano et al. (1980) NATURE 286:5774), and any other signal sequences which are known in the art (see, e.g., Watson (1984) NUCLEIC ACIDS RESEARCH 12:5145). Each of these references is incorporated by reference herein.

Signal sequences have been well characterized in the art and are known typically to contain 16 to 30 amino acid residues, and may contain greater or fewer amino acid residues. A typical signal peptide consists of three regions: a basic N-terminal region, a central hydrophobic region, and a more polar C-terminal region. The central hydrophobic region contains 4 to 12 hydrophobic residues that anchor the signal peptide across the membrane lipid bilayer during transport of the nascent polypeptide. Following initiation, the signal peptide is usually cleaved within the lumen of the endoplasmic reticulum by cellular enzymes known as signal peptidases. Potential cleavage sites of the signal peptide generally follow the “(−3, −1) rule”. Thus a typical signal peptide has small, neutral amino acid residues in positions −1 and −3 and lacks proline residues in this region. The signal peptidase will cleave such a signal peptide between the −1 and +1 amino acids. Thus, the signal sequence may be cleaved from the amino-terminus of the fusion protein during secretion. This results in the secretion of an Fc fusion protein consisting of the immunoglobulin Fc region and the target protein. A detailed discussion of signal peptide sequences is provided by von Heijne (1986) NUCLEIC ACIDS RES. 14:4683, the disclosure of which is incorporated by reference herein.

As would be apparent to one of skill in the art, the suitability of a particular signal sequence for use in the secretion cassette may require some routine experimentation. Such experimentation will include determining the ability of the signal sequence to direct the secretion of an Fc fusion protein and also a determination of the optimal configuration, genomic or cDNA, of the sequence to be used in order to achieve efficient secretion of Fc fusion proteins. Additionally, one skilled in the art is capable of creating a synthetic signal peptide following the rules presented by von Heijne, referenced above, and testing for the efficacy of such a synthetic signal sequence by routine experimentation. A signal sequence can also be referred to as a “signal peptide,” “leader sequence,” or “leader peptides.”

The fusion of the signal sequence and the immunoglobulin Fc region is sometimes referred to herein as secretion cassette. An exemplary secretion cassette useful in the practice of the invention is a polynucleotide encoding, in a 5′ to 3′ direction, a signal sequence of an immunoglobulin light chain gene and an Fcγ1 region of the human immunoglobulin γ1 gene. The Fcγ1 region of the immunoglobulin Fcγ1 gene preferably includes at least a portion of the immunoglobulin hinge domain and at least the CH3 domain, or more preferably at least a portion of the hinge domain, the CH2 domain and the CH3 domain. As used herein, the “portion” of the immunoglobulin hinge region is understood to mean a portion of the immunoglobulin hinge that contains at least one, preferably two cysteine residues capable of forming interchain disulfide bonds. The DNA encoding the secretion cassette can be in its genomic configuration or its cDNA configuration. Under certain circumstances, it may be advantageous to produce the Fc region from human immunoglobulin Fc-γ2 heavy chain sequences. Although Fc fusions based on human immunoglobulin γ1 and γ2 sequences behave similarly in mice, the Fc fusions based on the γ2 sequences can display superior pharmacokinetics in humans.

In another embodiment, the DNA sequence encodes a proteolytic cleavage site interposed between the secretion cassette and the target protein. A cleavage site provides for the proteolytic cleavage of the encoded fusion protein thus separating the Fc domain from the target protein. As used herein, “proteolytic cleavage site” is understood to mean amino acid sequences which are preferentially cleaved by a proteolytic enzyme or other proteolytic cleavage agents. Useful proteolytic cleavage sites include amino acids sequences which are recognized by proteolytic enzymes such as trypsin, plasmin or enterokinase K. Many cleavage site/cleavage agent pairs are known. See, for example, U.S. Pat. No. 5,726,044, the disclosure of which is incorporated herein by reference.

In the Examples disclosed herein, high levels of Fc-Leptin fusion proteins were produced. The initial clones produced about 50 μg/mL of Fc-Leptin, which could be purified readily to homogeneity by Protein A chromatography. Expression levels often can be increased several fold by subcloning. In addition, the Fc-Leptin fusion proteins could be cleaved and further purified, e.g., by affinity purification. As stated above, it is found that when leptin is expressed as Fc fusion molecules, high levels of expression are obtained, presumably because the Fc portion acts as a carrier, helping the polypeptide at the C-terminus to fold correctly and to be secreted efficiently. Moreover, the Fc region is glycosylated and highly charged at physiological pH, thus the Fc region can help to solubilize hydrophobic proteins.

In addition to the high levels of expression, leptin fusion proteins exhibited longer serum half-lives compared to leptin alone, due in part to their larger molecular sizes. For example, murine Fc-murine leptin has a circulating half-life of 8.8 hours in mouse, as compared to 18 minutes for murine leptin (see, Example 14 below). Leptin, having a molecular weight of about 16 kD, is small enough to be cleared efficiently by renal filtration. In contrast, the Fc-Leptin fusion protein has a molecular weight of about 90 kD since there are two leptin moieties each attached to an immunoglobulin Fc region, wherein the Fc regions are covalently bonded to one another. Such a dimeric structure should exhibit a higher binding affinity to the leptin receptor, the sequence of which suggests that it includes two ligand-binding domains (Tartaglia et al. (1995) CELL 83:1263). Since the leptin activity appears to be receptor-mediated, the leptin fusion proteins will be potentially more efficacious than leptin itself.

Additionally, many protein ligands are known to bind to their receptors as a dimer. If leptin belongs to the class of dimeric protein ligands, the physical constraint imposed by the immunoglobulin Fc region on leptin would make the dimerization an intramolecular process, thus, shifting the equilibrium in favor of the dimer and enhancing its binding to its receptor. Cysteine residues also can be introduced by standard recombinant DNA technology to the monomer at appropriate places to stabilize the dimer through covalent disulfide bond formation.

The fusion proteins of the invention provide several important clinical benefits. As demonstrated in the ob/ob mouse model, an intraperitoneal or subcutaneous injection of 0.1 mg/kg/day of murine leptin in the form of muFc-muLeptin was enough to achieve comparable reductions in body weight when compared with the 5 to 20 mg/kg/day of bacterially produced leptin (Pelleymounter et al. (1995) SCIENCE 269:540; Hallas et al. (1995) SCIENCE 269:543; Chebab et al. (1 996) NATURE GENETICS 12:318; Mounzih et al. (1997) ENDOCRINOLOGY 138:1190). The frequency of injection could be cut down to three times weekly if a dose of 0.25 mg/kg was used. Furthermore, ob/ob mice injected daily with 0.25 mg/kg muFc-muLeptin for over four months still responded favorably to the treatment, with no detectable side effects. Indeed the mice remained very healthy, with decreased appetite and increased thermogenesis and locomotor activities. In light of these results, the ability to construct the various structural conformations of Fc-Leptin of the invention provides molecules which may demonstrate improved efficacy over the native anti-obesity protein.

The fusion proteins of the invention when administered by injection at a dose of about 0.25 mg/kg/day for 5 days to an ob/ob mouse having an initial body weight of at least about 50 grams, induce about a 10% (about 5 gram), more preferably about a 12% (about 6 gram) or even more preferably about a 15% (about 7.5 gram) loss of the initial body weight. More preferably, the fusion proteins of the invention, when administered by injection at a dose of about 0.1 mg/kg/day for 5 days to an ob/ob mouse having an initial body weight of at least about 50 grams, induce about a 10% (about 5 gram), more preferably about a 12% % (about 6 gram), or even more preferably about a 15% (about 7.5 gram) loss of the initial body weight. Such dosages preferably result in a 10-20% reduction in body weight.

Another embodiment of the present invention provides constructs having various structural conformations, e.g., bivalent or multivalent constructs, dimeric or multimeric constructs, and combinations thereof. Such functional conformations of molecules of the invention allow the synergistic effect of leptin and other anti-obesity proteins to be explored in animal models.

The present invention also provides methods for the production of leptin of non-human species as Fc fusion proteins. Non-human leptin fusion proteins are useful for preclinical studies of leptin because efficacy and toxicity studies of a protein drug must be performed in animal model systems before testing in human beings. A human protein may, under certain circumstances, not work in a mouse model since the protein may elicit an immune response, and/or exhibit different pharmacokinetics thereby skewing the test results. Therefore, the equivalent mouse protein can, under certain circumstances, be a better surrogate for the human protein for testing in a mouse model.

The present invention provides methods of treating obesity and related conditions and causes thereof by administering the DNA, RNA or proteins of the invention to a mammal having such a condition. Related conditions may include, but are not limited to, diabetes, hypertension, heart disease, cancer and related disorders. In view of the broad roles played by leptin in modulating neuroendocrine responses (Freidman and Halaas (1998) NATURE 395:763), the present invention also provides methods for treating conditions alleviated by the administration of leptin. These methods include administering to a mammal having the condition, which may or may not be directly related to obesity, an effective amount of a composition of the invention.

The proteins of the invention not only are useful as therapeutic agents, but one skilled in the art recognizes that the proteins are useful in the production of antibodies for diagnostic use. Likewise, appropriate administration of the DNA or RNA, for example, in a vector or other delivery system for such uses, is included in methods of use of the invention. Furthermore, the constructs of the invention are useful for controlling weight for cosmetic purposes in mammals. A cosmetic purpose seeks to control the weight of a mammal to improve bodily appearance. The mammal is not necessarily obese. Such cosmetic use forms part of the present invention. In addition, use of Fc-Leptins derived from other mammals, e.g., bovine and porcine, are useful for raising lean animals for meat.

It is not known if the Fc-Leptin fusion protein can cross the blood-brain barrier to reach the receptor in the hypothalamus. If the Fc-Leptin fusion protein does not cross the blood-brain barrier, then its superior efficacy as an anti-obesity agent suggests a new mechanism of action or that there are leptin receptors outside the brain. As a fusion protein with the immunoglobulin Fc region, Fc-Leptin fusion protein may have a very favorable tissue distribution and a slightly different mode of action to achieve clinical efficacy and even overcome leptin resistance especially in view of its long serum half-life and the high dose of soluble protein that can be administered. Data from subcutaneous injections in mice suggest that intramuscular injections in humans should be equally successful. It may also be desirable to administer Fc-Leptin fusion protein as a nasal spray, inhaled preparation, dermal patch or eye drop. If the Fc-Leptin fusion protein is to be administered as an inhaled preparation, it is useful to formulate the fusion protein so that it is aggregated into small particles that can undergo trans-cytosis across the lung epithelia.

The DNA constructs (or gene constructs) of the invention also can be used as a part of a gene therapy protocol to deliver nucleic acids encoding leptin or a fusion protein construct thereof. The invention features expression vectors for in vivo transfection and expression of leptin or a fusion protein construct thereof in particular cell types so as to reconstitute or supplement the function of leptin. Expression constructs of leptin, or fusion protein constructs thereof, may be administered in any biologically effective carrier, e.g. any formulation or composition capable of effectively delivering the leptin gene or fusion protein construct thereof to cells in vivo. Approaches include insertion of the subject gene in viral vectors including recombinant retroviruses, adenovirus, adeno-associated virus, and herpes simplex virus-1, or recombinant bacterial or eukaryotic plasmids.

It is contemplated that the compositions of the present invention may be provided to an animal by any suitable means, directly (e.g., locally, as by injection, implantation or topical administration to a tissue locus) or systemically (e.g., parenterally or orally). Where the composition is to be provided parenterally, such as by intravenous, subcutaneous, ophthalmic, intraperitoneal, intramuscular, buccal, rectal, vaginal, intraorbital, intracerebral, intracranial, intraspinal, intraventricular, intrathecal, intracisternal, intracapsular, intranasal or by aerosol administration, the composition preferably comprises part of an aqueous or physiologically compatible fluid suspension or solution. Thus, the carrier or vehicle is physiologically acceptable so that in addition to delivery of the desired composition to the patient, it does not otherwise adversely affect the patient's electrolyte and/or volume balance. The fluid medium for the agent thus can comprise normal physiologic saline.

Preferred dosages per administration of the fusion proteins of the invention are within the range of 50 ng/m² to 1 g/m², more preferably 5 μg/m² to 200 mg/m², and most preferably 100 μg/m² to 10 mg/m². Preferred dosages per administration of nucleic acids encoding the fusion proteins of the invention are within the range of 1 μg/m² to 100 mg/m², more preferably 20 μg/m² to 10 mg/m², and most preferably 400 μg/m² to 4 mg/m². It is contemplated, however, that the optimal modes of administration, and dosages may be determined by routine experimentation well within the level of skill in the art.

The invention is illustrated further by the following non-limiting examples.

### EXAMPLES

### Example 1

Expression Of muFc-muLeptin

A sample of mRNA was prepared from the fat cells of a normal C57/BL6 mouse and the mRNA reverse transcribed using reverse transcriptase. The resultant cDNA was used as template for a polymerase chain reaction (PCR) to clone and adapt the murine leptin cDNA for expression as a muFc-muLeptin fusion protein. The forward primer was 5′ C CCG GGT AAA GTG CCT ATC CAG AAA GTC C (SEQ ID NO: 9), where the sequence CCCGGG (XmaI restriction site) followed by TAAA encodes the carboxy terminus of the immunoglobulin heavy chain. The sequence in bold encodes the N-terminus of murine leptin. The reverse primer was 5′ CTC GAG TCA GCA TTC AGG GCT AAC ATC (SEQ ID NO: 10), which encodes the C-terminal sequence of leptin with its translation STOP codon (anticodon, TCA), and this was followed by an XhoI site (CTCGAG). The resulting 450 base-pair PCR product was cloned and sequenced. Sequence analysis confirmed that the product encoded mature murine leptin adapted for expression, i.e., with a XmaI site at its 5′ end and a XhoI site at its 3′ end.

The expression vector pdCs-muFc-muLeptin was constructed as follows. The XmaI-XhoI restriction fragment containing the murine leptin cDNA was then ligated to the XmaI-XhoI fragment of the pdCs-muFc vector according to Lo et al. (PROTEIN ENGINEERING (1998) 11:495). muFc is the murine Fc fragment of the murine immunoglobulin γ2a. The resultant vector, pdCs-muFc-muLeptin, was used to transfect mammalian cells for the expression of muFc-muLeptin.

### Example 2

Transfection and Expression of Protein

For transient transfection, the plasmid was introduced into human kidney 293 cells by coprecipitation of plasmid DNA with calcium phosphate (Sambrook et al. (1989) “Molecular Cloning—A Laboratory Manual,” Cold Spring Harbor, N.Y.) or by lipofection using Lipofectamine Plus (Life Technologies, Gaithersburg, Md.) in accordance with manufacturer's instructions.

In order to obtain stably transfected clones, plasmid DNA was introduced into the mouse myeloma NS/0 cells by electroporation. NS/0 cells were grown in Dulbecco's modified Eagle's medium supplemented with 10% fetal bovine serum, 2 mM glutamine and penicillin/strepomycin. About 5×10⁶ cells were washed once with PBS and resuspended in 0.5 ml PBS. Ten μg of linearized plasmid DNA then was incubated with the cells in a Gene Pulser Cuvette (0.4 cm electrode gap, BioRad) on ice for 10 min. Electroporation was performed using a Gene Pulser (BioRad, Hercules, Calif.) with settings at 0.25 V and 500 μF. Cells were allowed to recover for 10 min. on ice, after which they were resuspended in growth medium and then plated onto two 96 well plates. Stably transfected clones were selected by growth in the presence of 100 nM methotrexate (MTX), which was introduced two days post-transfection. The cells were fed every 3 days for two to three more times, and MTX-resistant clones appeared in 2 to 3 weeks. Supernatants from clones were assayed by anti-Fc ELISA to identify high producers. High producing clones were isolated and propagated in growth medium containing 100 nM MTX.

For routine characterization by gel electrophoresis, Fc fusion proteins in the conditioned media were captured on Protein A Sepharose (Repligen, Cambridge, Mass.) and then eluted by boiling in the protein sample buffer with or without 2-mercaptoethanol. After fractionization by SDS-polyacrylamide gel electrophoresis (SDS-PAGE), the protein bands were visualized by Coomassie staining. muFc-muLeptin had an apparent MW of about 50 kD via SDS-PAGE.

For purification, the fusion proteins were bound to Protein A Sepharose followed by elution in a sodium phosphate buffer (100 mM NaH₂PO₄, pH 3, and 150 mM NACl). The eluate was then immediately neutralized with 0.1 volume of 2 M Tris-hydrochloride, pH 8.

### Example 3

ELISA Procedures

ELISAs were used to determine the concentrations of protein products in the supernatants of MTX-resistant clones and other test samples. The amounts of human Fc- and murine Fc-containing proteins were determined by the anti-huFc ELISA and the anti-muFc ELISA, respectively.

The anti-huFc ELISA is described in detail below:

A. Coating Plates.

ELISA plates were coated with AffiniPure Goat anti-Human IgG (H+L) (Jackson Immuno Research Laboratories, West Grove, Pa.) at 5 μg/mL in PBS, and 100 μL/well in 96-well plates (Nunc-Immuno plate Maxisorp). Coated plates were covered and incubated at 4° C. overnight. Plates then were washed 4 times with 0.05% Tween (Tween 20) in PBS, and blocked with 1% BSA/1% goat serum in PBS, 200 μL/well. After incubation with the blocking buffer at 37° C. for 2 hrs, the plates were washed 4 times with 0.05% Tween in PBS and tapped dry on paper towels.

B. Incubation With Test Samples and Secondary Antibody

Test samples were diluted to the proper concentrations in sample buffer, which contains 1% BSA/1% goat serum/0.05% Tween in PBS. A standard curve was prepared with a chimeric antibody (with a human Fc), the concentration of which was known. To prepare a standard curve, serial dilutions are made in the sample buffer to give a standard curve ranging from 125 ng/mL to 3.9 ng/mL. The diluted samples and standards were added to the plate, 100 μL/well, and the plate incubated at 37° C. for 2 hr. After incubation, the plate was washed 8 times with 0.05% Tween in PBS. To each well was then added 100 μL of the secondary antibody, the horseradish peroxidase-conjugated anti-human IgG (Jackson Immuno Research), diluted around 1:120,000 in sample buffer. The exact dilution of the secondary antibody has to be determined for each lot of the HRP-conjugated anti-human IgG. After incubation at 37° C. for 2 hr, the plate was washed 8 times with 0.05% Tween in PBS.

C. Development

The substrate solution was added to the plate at 100 μL/well. The substrate solution was prepared by dissolving 30 mg of OPD (o-phenylenediamine dihydrochloride, 1 tablet) into 15 mL of 0.025 M Citric acid/0.05 M Na₂HPO₄ buffer, pH to 5, which contained 0.03% of freshly added H₂O₂. The color was allowed to develop for 30 min. at room temperature in the dark. The developing time is subject to change, depending on lot to lot variability of the coated plates, the secondary antibody, etc. Watch the color development in the standard curve to determine when to stop the reaction. The reaction was stopped by adding 4N H₂SO₄, 100 μL/well. The plate was read by a plate reader, which was set at both 490 and 650 nm and programmed to subtract the background OD at 650 nm from the OD at 490 nm.

The procedure for the anti-muFc ELISA was similar, except that ELISA plate was coated with AffiniPure Goat anti-murine IgG (H+L) (Jackson Immuno Research) at 5 μg/mL in PBS, and 100 μL/well; and the secondary antibody was horseradish peroxidase-conjugated goat anti-muIgG (Southern Biotechnology Assoc., Birmingham, Ala.), used at 1 in 5000 dilution.

### Example 4

Expression of huFc-huLeptin

Human Fat Cell Quick-Clone cDNA (Clontech, Palo Alto, Calif.) was used as a template for PCR to clone and adapt human leptin cDNA for expression as a huFc-huLeptin fusion protein. The forward primer was 5′ C CCG GGT AAA GTG CCC ATC CAA AAA GTC CA (SEQ ID NO: 11), where the sequence C CCG GG T AAA (SEQ ID NO: 12) encodes the carboxy terminus of the immunoglobulin heavy chain, followed by sequence (in bold) encoding the mature N-terminus of leptin. The C CCG GG sequence is an XmaI restriction site introduced by silent mutation (Lo et al., (1998) PROTEIN ENGINEERING 11:495). The reverse primer was 5′ CTC GAG TCA GCA CCC AGG GCT GAG GTC (SEQ ID NO: 13), which encodes the anti-sense sequence of the carboxyl terminus of leptin with its translation STOP codon (anticodon, TCA), and this was followed by an XhoI site (CTCGAG). The resulting 450 base-pair PCR product was cloned and sequenced. Sequence analysis confirmed that the product encoded mature human leptin adapted for expression, i.e., with an XmaI site at its 5′ end and a XhoI site at its 3′ end.

The expression vector pdCs-huFc-huLeptin was constructed as follows. The XmaI-XhoI restriction fragment containing the human leptin cDNA was ligated to the XmaI-XhoI fragment of the pdCs-huFc vector according to Lo et al. (PROTEIN ENGINEERING (1998)11:495). huFc is the human Fc fragment of the human immunoglobulin ≢51. The resultant vector, pdCs-huFc-huLeptin, was used to transfect mammalian cells for the expression of huFc-huLeptin.

### Example 5

Construction of Expression Vectors for muLeptin-muFc and muLeptin-Gly-Ser-linker-muFc

Murine leptin cDNA was adapted for expression as a muLeptin-muFc fusion protein by PCR. The forward primer, 5′ C TTA AG C GTG CCT ATC CAG AAA GTC CA (SEQ ID NO: 14), introduced an AfIII (CTTAAG) site for ligating the cDNA sequence encoding the mature N-terminus of murine leptin (sequence in bold) to the DNA encoding the signal peptide. The reverse primer, 5′ GATATC GCA TTC AGG GCT AAC ATC (SEQ ID NO: 15), introduced an EcoRV site immediately downstream of the sequence encoding the carboxyl terminus of the murine leptin without the STOP codon (anti-sense sequence in bold). The EcoRV site served as a linker-adaptor for an inframe fusion of the murine leptin to the murine Fc, as discussed below. The resulting 450 base-pair PCR product was cloned and completely sequenced. The AfIII-EcoRV fragment encoding the mature murine leptin was then used for construction of the pdCs-muLeptin-muFc expression vector.

The ligation product of the AfIII-EcoRV fragment encoding the mature murine leptin and the XbaI-AfIII fragment encoding the signal peptide of an immunoglobulin light chain (Lo et al. (1998) PROTEIN ENGINEERING 11:495) was subcloned. The resultant XbaI-EcoRV fragment encodes the signal peptide followed by the mature murine leptin without the STOP codon.

To adapt an EcoRV site to the 5′ end of the muFc DNA, the ligation product of the AfIII-XhoI fragment encoding murine Fc (Lo et al. (1998) PROTEIN ENGINEERING 11:495) and the following linker-adaptor were subcloned into an EcoRI-XhoI cloning vector.

EcoRI Sticky End

| | |
|---|---|
| EcoRI sticky end | |
| 5′ AATTC GAT ATC | (SEQ ID NO: 16) |
| 3′ G CTA TAG AATT | (SEQ ID NO: 17) |
| AfIII sticky end | |

AfIII Sticky End

The foregoing linker-adaptor contains EcoRI and AfIII sticky ends, and it also contains an EcoRV site (GATATC). After subcloning, an EcoRV-XhoI fragment encoding the muFc fragment with a STOP codon was isolated. This fragment then was ligated with the XbaI-EcoRV fragment encoding the signal peptide and the mature murine leptin (described above) and the XbaI-XhoI digested pdCs vector fragment. The resultant expression plasmid, designated pdCs-muLeptin-muFc, was used for transfection of mammalian cells.

For the construction of pdCs-muLeptin-Gly-Ser-linker-muFc, the pdCs-muLeptin-muFc DNA was linearized at the unique EcoRV site, and the following unphosphorylated linker was inserted by ligation:

(SEQ ID NO: 18)

5′ GGC GCA GGA GGT TCT GGC GGA TCC 3′

(SEQ ID NO: 19)

3′ CCG CGT CCT CCA AGA CCG CCT AGG 5′

The correct construction was confirmed by DNA sequencing to ensure that the correct linker sequence had been inserted in the proper orientation. The resultant vector, pdCs-muLeptin-Gly-Ser-linker-muFc, was used for transfection of mammalian cells.

### Example 6

Reduced Levels of Expression for muLeptin-muFc and muLeptin-Gly-Ser-linker-muFc

Since the C-terminal cysteine residue of leptin is involved in intramolecular disulfide bonding with cysteine-117, this may pose a problem in protein folding and subsequent secretion if leptin is made as a leptin-Fc fusion protein. To test if this is indeed the case, expression vectors for muLeptin-muFc and muLeptin-Gly-Ser linker-muFc were constructed as described in Example 5. The latter construct encodes a flexible linker rich in glycine and serine residues interposed between leptin and Fc so as to allow more freedom for the leptin to form the disulfide bond and fold correctly. Transient expression in 293 cells was analyzed by anti-muFc ELISA, and Western blot analysis using both anti-muFc antibody (horseradish peroxidase-conjugated goat anti-muIgG, Fcγ, from Jackson ImmunoResearch) and anti-muLeptin antibody (biotinylated anti-mouse leptin polyclonal antibody, from R & D Systems, Minneapolis, Minn.). Very low levels of expression were detected in the supernatants of each construct. Analysis of total cell lysates showed that the majority of the muLeptin-muFc and muLeptin-Gly-Ser linker-muFc stayed inside the cells. Stable NS/0 clones also were isolated. The expressed levels of muLeptin-muFc (with or without linker) were at most about I()% that of mul-c-muLeptin.

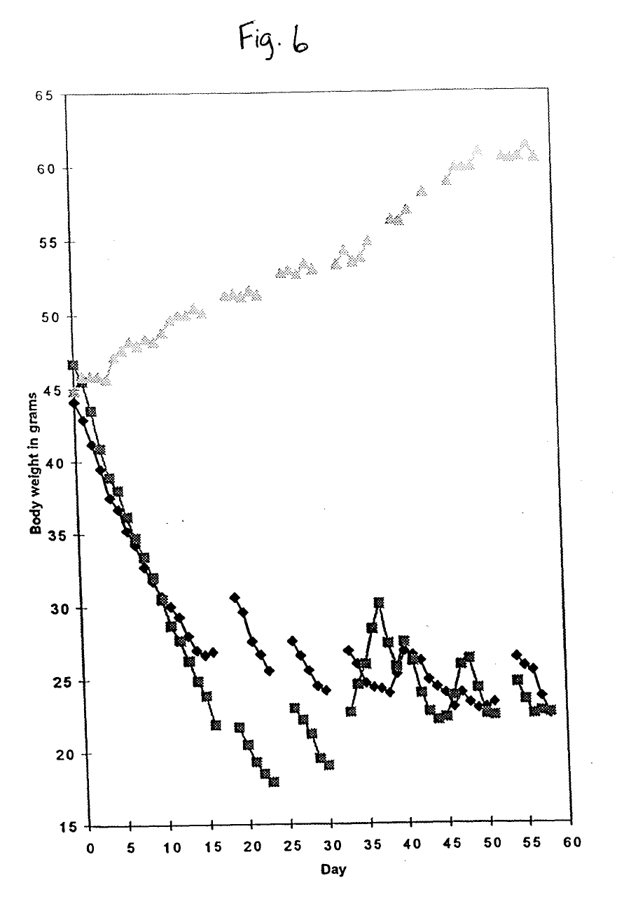

Furthermore, subsequent studies suggest that the Leptin-Fc fusion protein was not as active in vivo as the Fc-Leptin fusion protein (see, FIG. 6). When ob/ob mice were injected intraperitoneally with Leptin-Fc at 0.25 mg/kg/day, no significant weight loss was observed. It is surprising that Fc-Leptin was more effective that Leptin-Fc, because these fusion proteins contain the same moieties and differ only in the order of the moieties in each polypeptide chain.

### Example 7

Treatment of ob/ob Mice by Intraperitoneal (IP) Injection of muFc-muLeptin

Five- to six-week old C57BL/6J ob/ob^{1J} mice, which were homozygous for the obese gene mutation (ob/ob mice), were purchased from Jackson Laboratories, Barr Harbor, Me. Two mice per group received either muFc-muLeptin or only PBS. muFc-muLeptin was dissolved in PBS and administered by daily (daily for the first 12 days; and only Monday through Friday thereafter) intraperitoneal injections. The amount of leptin injected was normalized to 0.25 mg of leptin per kg body weight of mouse. The control group received PBS only. All mice were allowed ad libitum access to food and water and the body weight was measured daily before the injection.

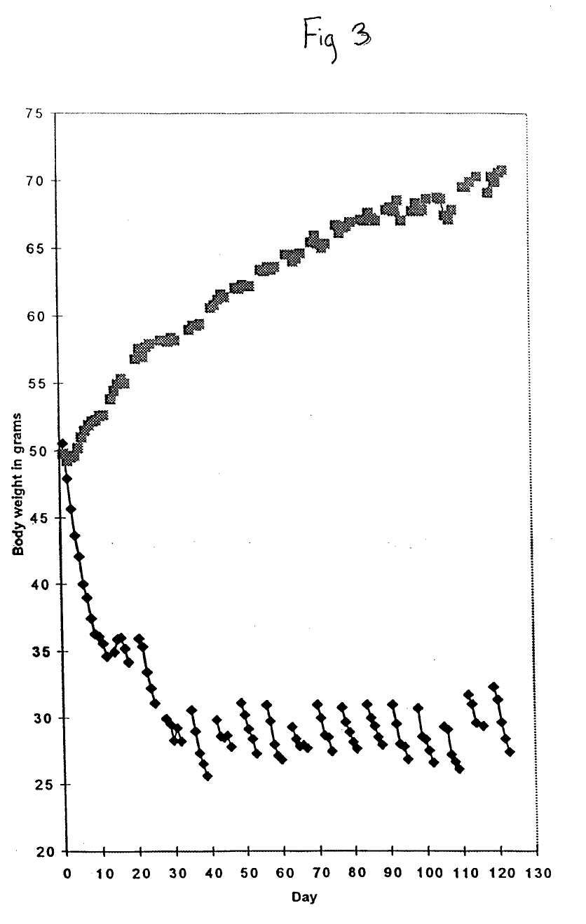

Over a 4 month period, the control group (squares in FIG. 3) had a steady increase of 40% in body weight (from 50 g to 70 g). The group receiving daily intraperitoneal injection of muFc-muLeptin had a 45% reduction in body weight (from 50.5 g to 28 g) over the first month, after which the body weight stabilized at about 27-31 g (diamonds in FIG. 3). Since the mice did not receive treatment over the weekends, their body weights increased to over 30 g by Mondays, but the daily treatment caused a steady decrease in body weight to about 27-28 g by Fridays. As shown in FIG. 3, muFc-muLeptin was shown to be effective for over 4 months.

Note that during the first two weeks of treatment, food intake was below the detection limit. After 3 to 4 weeks, when the body weights had decreased to about 30 g and the adipose tissue apparently was depleted, the mice consumed an average of about 3 g of food per mouse daily. This is consistent with the results of Mounzih et al. (Mounzih et al (1997) ENDOCRINOLOGY 138:1190), which showed that food consumption of ob/ob mice receiving leptin treatment at 20 mg/kg resumed to approximately 2.6-3.2 g at day 45.

### Example 8

Treatment of ob/ob Mice by Subcutaneous (SC) Injection of muFc-muLeptin

Subcutaneous injection of muFc-muLeptin was found to be as effective as intraperitoneal injection in reducing body weight of ob/ob mice. Five to six week old ob/ob mice (3 mice per group) were treated with muFc-muLeptin by daily (Monday through Friday only) SC injection. The amounts of leptin injected were normalized to 0.25 or 0.1 mg of leptin per kg body weight of mouse. All mice were allowed ad libitum access to food and water and the body weight was measured daily before the injection. After 17 days, the mice receiving 0.1 and 0.25 mg of leptin/kg had a reduction of 14% and 22% in body weight, respectively, while the control group receiving PBS had a 15% weight gain. The decrease in food intake in mice receiving SC injections is similar to that in mice receiving IP injections of equivalent doses.

### Example 9

Treatment of ob/ob Mice by Intravenous (IV) Injection of muFc-muLeptin

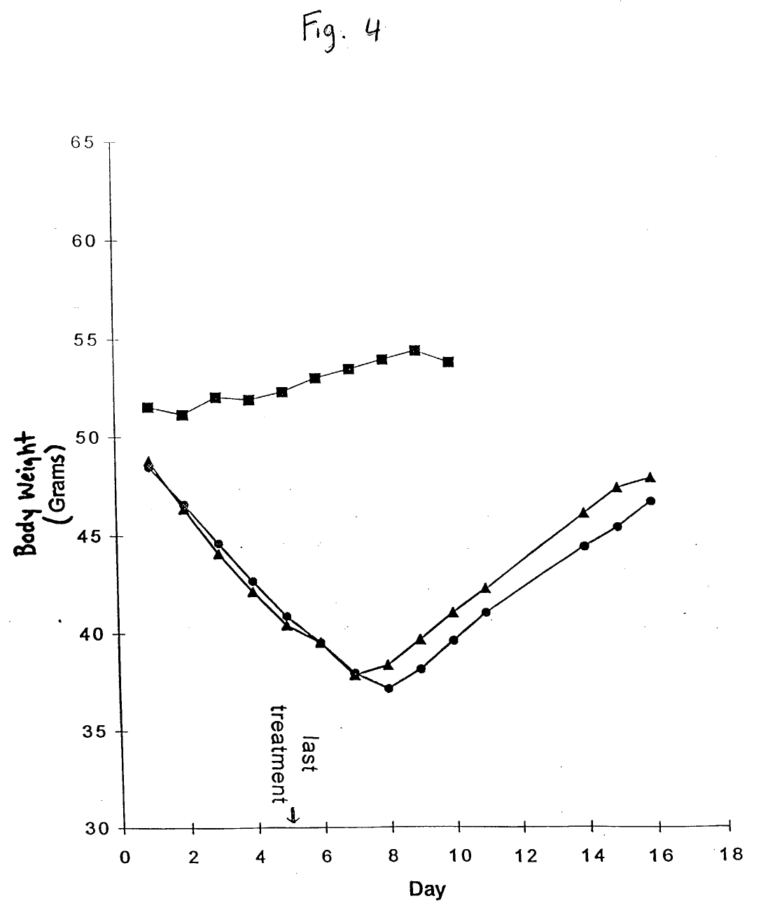

Intravenous (IV) injection of muFc-muLeptin was found to be equally effective in reducing body weight in ob/ob mice. Ob/ob mice (2 mice per group) were treated with daily IV injections of muFc-muLeptin at 0.25 or 1 mg of leptin per kg or PBS. All mice were allowed ad libitum access to food and water and the body weight was measured daily before the injection. Treatment was stopped after 5 days, but the body weight continued to be recorded daily. As shown in FIG. 4, treatment with 0.25 and 1 mg/kg of leptin as muFc-muLeptin (triangles and circles, respectively) caused the body weight to decrease for the next 48 and 72 hrs, respectively. These results suggest that muFc-muLeptin has a much longer circulating half-life than murine leptin, based on the high, frequent doses of leptin needed for reducing body weight.

### Example 10

Treatment of ob/ob Mice with muFc-muLeptin 3 Times Weekly or Once Every 4 Days

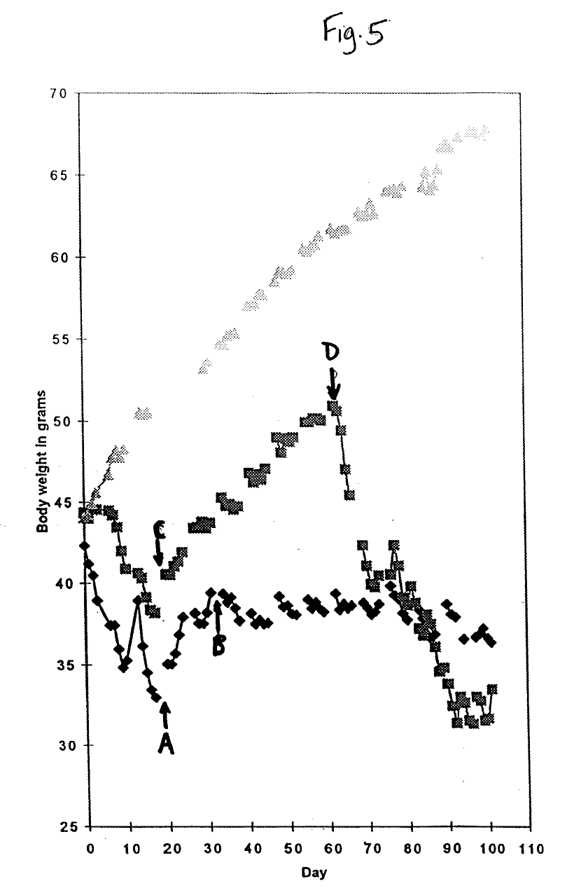

FIG. 5 shows the effect of different dosing schedules on the body weight of ob/ob mice. Specifically, a group of 3 ob/ob mice (solid diamonds) received 0.25 mg/kg of murine leptin in the form of muFc-muLeptin by SC injections daily from Monday through Friday up to point A; from point A to point B the frequency of injection was reduced to Monday and Friday only; thereafter, the frequency of injection was increased to 3 times weekly (Monday, Wednesday, and Friday). Another group, also consisting of 3 ob/ob mice (squares), received 0.1 mg/kg of murine leptin in the form of muFc-muLeptin by SC injections daily from Monday to Friday up to point C; from point C to point D the frequency of injection was reduced to 3 times weekly (Monday, Wednesday, and Friday); after point D, however, the dosage was increased to 1 mg/kg once every 4 days. A control group of 3 ob/ob mice (triangles) received PBS daily, Monday through Friday. All mice were allowed ad libitum access to food and water and the body weight was measured daily before the injection.

As shown in FIG. 5, 0.25 mg/kg of muFc-muLeptin injected SC three times a week (Monday, Wednesday, and Friday) was effective in stabilizing the body weight at about 36 to 39 g for over 9 weeks, and 1 mg/kg injected SC once every 4 days resulted in a reduction from 51 g to 34 g in 4 weeks, after which the body weight stabilized at between 30 to 33 g. A dosing schedule of 0.1 mg/kg 3 times weekly was ineffective in reducing body weight. These results suggest that daily injections of muFc-muLeptin are unnecessary given its long lasting effect when injected at an appropriate dose.

### Example 11

Treatment of Lean Mice and db/db Mice With muFc-muLeptin

For comparison with ob/ob mice, normal C57BL/6J, C57BL/KS and Balb/C mice, and diabetic C57BL/KS db/db mice (all were purchased from Jackson Laboratories, Barr Harbor, Me.) all received daily (Monday through Friday) intraperitoneal injection or subcutaneous injection of muFc-muLeptin in PBS. The amounts of leptin injected were normalized to 0.25 mg or 1 mg of leptin per kg body weight of mouse. As shown in Table 1, muFc-muLeptin at both dosage levels had no effect on db/db mice, which lack the receptor for leptin. On normal C57BL/6J, C57BL/KS and Balb/C mice, the low dose had a very modest effect. However, the high dose resulted in a significant reduction of body weight over 19 days (Table 1), independent of the age of the mice.

TABLE 1
Percentage change in body weight of mice (3 mice per group) treated
with 0, 0.25 or 1 mg/kg of muFc-muLeptin by daily
(Monday through Friday) intraperitoneal (IP)
or subcutaneous (SC) injections for 19 days.

| | Route | Age | Vehicle | 0.25 mg/kg | 1 mg/kg |
|---|---|---|---|---|---|
| ob/ob | IP | 2 mo. | +14.7 | −23.3 | −17.4** |
| db/db | SC | 2 mo. | +7.21 | +6.78 | +5.01 |
| db/db | IP | 5 mo. | +1.82 | +5.66 | +5.28 |
| C57BL/6J | IP | 5 mo. | +1.03 | −1.69 | −13.9 |
| C57BL/KS | IP | 5 mo. | +0.22 | −0.13 | −16.9 |
| Balb/C | IP | 2 mo. | +9.18 | −5.4 | −9.19 |

**Treatment of ob/ob mice at 1 mg/kg was stopped after 5 days because the lower dose of 0.25 mg/kg was found to be just as effective.

### Example 12

Treatment of ob/ob Mice by Intraperitoneal (IP) Injection of huFc-huLeptin

huFc-huLeptin was administered by IP instead of SC to reduce immunogenicity in mice. One ob/ob mouse received 0.1 mg/kg of human leptin in the form of huFc-huLeptin by IP injections daily (for the first 17 days, and thereafter only Monday through Friday). Another ob/ob mouse received a higher dose of 0.5 mg/kg daily (for the first 17 days, and thereafter only Monday through Friday) until day 33, after which the frequency of injection was reduced to 3 times weekly (Monday, Wednesday, and Friday). A control ob/ob mouse received PBS daily (for the first 17 days, and thereafter only Monday through Friday). All mice were allowed ad libitum access to food and water and the body weight was measured daily before the injection.

FIG. 6 shows that huFc-huLeptin was as effective as muFc-muLeptin in reducing body weight in ob/ob mice. Another group of two older ob/ob mice received an intermediate dose of 0.25 mg/kg daily (for the first 10 days, and thereafter only Monday through Friday). Their body weight decreased from 65 g to 31 g (−51.4%) in 23 days, after which their body weight fluctuated between about 31 g on Mondays to about 26 g on Fridays (data not shown). It is remarkable that after almost two months of treatment, huFc-huLeptin maintained its efficacy and did not seem to be adversely affected by any immunologic response that might have developed against the human protein.

This experiment has been repeated with larger groups of mice (n=8). In addition, ob/ob mice have been treated for over 15 months with Fc-Leptin with the result that the weight of the mice was maintained in the range of 20-30 grams. Over this period of time, the mice suffered no apparent adverse side effects.

Additional experiments also indicated that daily administration of Fc-Leptin by intraperitoneal injection, subcutaneous injection, and intravenous injection all yielded similar results. Thus, the route of injection does not appear to be important when quantitating Fc-Leptin in vivo activity in ob/ob mice.

### Example 13

Treatment of Infertility in ob/ob Mice by Intraperitoneal (IP) Injection of muFc-muLeptin

ob/ob males and ob/ob females were treated with muFc-muLeptin by daily IP injections of 0.25 mg/kg. Each ob/ob male was initially housed with one ob/ob female and one normal C57BL/6J female. When there was a rapid increase in body weight indicative of pregnancy, the pregnant mouse was isolated. After about 2 to 4 weeks of treatment, all six ob/ob males had their infertility defect corrected and impregnated normal and/or ob/ob females. All normal C57BL/6J mothers delivered and nursed their pups normally. Of the six pregnant ob/ob females, only four had normal deliveries, leading to homozygous ob/ob pups. However, none of the pups survived beyond the first day because the ob/ob mothers did not lactate normally.

### Example 14

Pharmacokinetics

The pharmacokinetics of muFc-muLeptin and murine leptin (R & D Systems, Minneapolis, Minn.) were compared. Ob/ob mice (6 mice per group) were injected in the tail vein. The amounts of leptin injected were normalized to 1 mg of leptin per kg body weight of mouse. Blood was obtained by retro-orbital bleeding immediately after injection (0 min), and at 0.1, 0.5, 1, 2, 4, 8, 24, and 48 hr post injection. Blood samples were collected in tubes containing heparin to prevent clotting. Cells were removed by centrifugation in an Eppendorf high-speed microcentrifuge for 4 min. The concentration of mouse leptin in the plasma was measured by using a mouse leptin immunoassay kit (R & D Systems, Minneapolis, Minn.). The circulating half-lives of muFc-muLeptin and murine leptin were determined to be 8.8 hr and 18 min, respectively.

Similarly, huFc-huLeptin was found to have a circulating half-life of over 10 hr in mice.

### Example 15

Construction of huFc(N→Q mutation)-huLeptin

In order to test whether N-linked glycosylation of the immunoglobulin Fc region affects the serum half-life of huFc-huLeptin, a recombinant huFc-huLeptin mutant was produced where the asparagine residue in a glycosylation site of the Fc region was mutated to a glutamine. Briefly, the only N-glycosylation site (Asn-Ser-Thr) encoded in the huFc-huLeptin DNA was mutated by PCR using the forward primer 5′ GAG CAG TAC CAA AGT ACG TAC CGT GTG GTC AGC (SEQ ID NO: 16) and reverse primer 5′ ACG GTA CGT ACT TTG GTA CTG CTC CTC CCG CG (SEQ ID NO: 17). The primers encoded the change from Asn-Ser-Thr to Gln (CAA)-Ser-Thr, which is no longer a site for N-glycosylation. In addition, the primers introduced a SnaBI site (TACGTA) by silent mutation to facilitate screening for the Asn to Gln (N to Q) mutation. Following mutagenesis by PCR, the SacII-SmaI fragment containing the N to Q substitution was confirmed by DNA sequencing, and then used to replace the corresponding fragment in pdCs-huFc-huLeptin to generate pdCs-huFc(N→Q)-huLeptin.

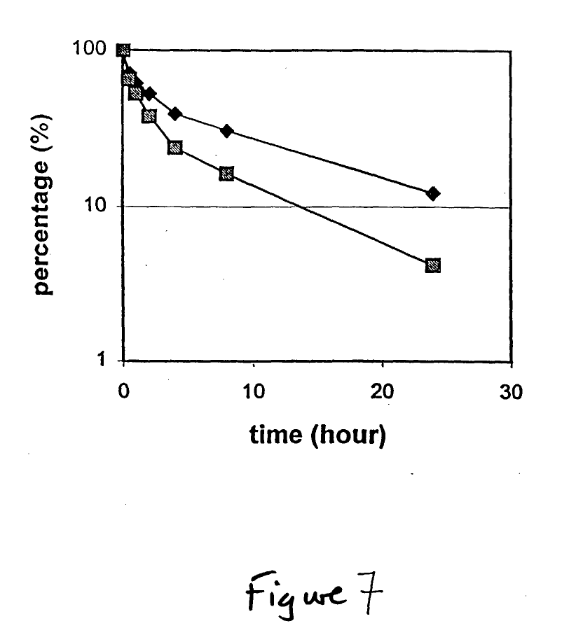

The expression plasmid pdCs-huFc(N→Q)-huLeptin was transfected into mammalian cells as described in Example 2. The purified huFc(N∝3Q)-huLeptin protein was then used for pharmacokinetic studies as described in Example 14. For direct comparison, equal amounts of huFc-huLeptin (1 mg of leptin/kg) or huFc(N∝3Q)-huLeptin (1 mg leptin/kg) were injected into mice in parallel. The concentrations of huFc(N→Q)-huLeptin and huFc-huLeptin in the mouse serum were measured by anti-huFc ELISA as described in Example 3. The results shown in FIG. 7 show that the huFc-huLeptin (diamonds) had a longer circulating half-life than huFc(N→Q)-huLeptin (squares).

Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

What is claimed is:

1. A nucleic acid encoding a fusion protein comprising:
(a) a signal sequence;
(b) an immunoglobulin Fc region; and
(c) a target protein sequence comprising leptin.

2. The nucleic acid of claim 1 wherein said signal sequence, said immunoglobulin Fc region and said target protein sequence are encoded serially in a 5′ to 3′ direction.

3. The nucleic acid of claim 1 wherein said signal sequence, said target sequence, and said immunoglobulin Fc region are encoded serially in a 5′ to 3′ direction.

4. The nucleic acid of claim 1 wherein said immunoglobulin Fc region comprises an immunoglobulin hinge region.

5. The nucleic acid of claim 1 wherein said immunoglobulin Fc region comprises an immunoglobulin hinge region and an immunoglobulin constant heavy chain domain.

6. The nucleic acid of claim 1 wherein said immunoglobulin Fc region comprises a hinge region and a CH3 domain.

7. The nucleic acid of claim 1 wherein said immunoglobulin Fc region lacks at least the CH1 domain.

8. The nucleic acid of claim 1 wherein said immunoglobulin Fc region encodes at least a portion of immunoglobulin γ.

9. A replicable expression vector for transfecting a mammalian cell, said vector comprising the nucleic acid of claim 1.

10. A mammalian cell harboring the nucleic acid of claim 1.

11. A fusion protein comprising an immunoglobulin Fc region and a target protein comprising leptin, wherein the fusion protein, when administered at a dose of about 0.25 mg/kg/day for 5 days to an ob/ob mouse having an initial body weight of at least about 50 grams, induces a 10% or 5 gram loss in body weight.

12. The fusion protein of claim 11, wherein the fusion protein, when administered at a dose of about 0.1 mg/kg/day, induces a 10% or 5 gram loss in body weight.

13. The fusion protein of claim 11 wherein the target protein comprises an amino acid sequence set forth in SEQ ID NO: 2 or 4.

14. The fusion protein of claim 11 wherein the leptin said target protein comprises at least two leptin molecules, wherein said two leptin molecules are linked by a peptide linker.

15. The fusion protein of claim 11 wherein said target protein is linked to an N-terminal end of said immunoglobulin Fc region.

16. The fusion protein of claim 11 wherein said target protein is linked to a C-terminal end of said immunoglobulin Fc region.

17. The fusion protein of claim 11 further comprising a peptide linker linking said immunoglobulin Fc region to said target protein.

18. A multimeric protein comprising at least two fusion proteins of claim 11 linked via a covalent bond.

19. The protein of claim 18, wherein the covalent bond is a disulfide bond.

20. A multimeric protein comprising at least two fusion proteins of claim 11 linked via a covalent bond.

21. The protein of claim 20, wherein the covalent bond is a disulfide bond.

22. The fusion protein of claim 11 wherein said immunoglobulin Fc region is glycosylated at least one glycosylation site.

23. A method of producing a fusion protein comprising the steps of
(a) providing the mammalian cell of claim 10; and
(b) culturing the mammalian cell to produce said fusion protein.

24. The method of claim 23 comprising the additional step of collecting said fusion protein.

25. The method of claim 23 comprising the additional step of purifying said fusion protein.

26. The method of claim 23 comprising the additional step of cleaving said immunoglobulin Fc region from said target protein.

27. The method of claim 26 comprising the additional step of cleaving said target protein at an internal cleavage site with a proteolytic enzyme endogenous to the mammalian cell.

28. A method of treating a condition alleviated by the administration of leptin comprising administering a nucleic acid of claim 1 to a mammal having said condition.

29. A method of treating a condition alleviated by the administration of leptin comprising administering a vector of claim 9 to a mammal having said condition.

30. A method of treating a condition alleviated by the administration of leptin comprising administering the fusion protein of claim 11 to a mammal having said condition.

31. A method of treating a condition alleviated by the administration of leptin comprising administering the multimeric protein of claim 18 to a mammal having said condition.
